(19) United States
(12) Patent Application Publication
(10) Pub. No.: US 2026/0306762 A1
(43) Pub. Date: Oct. 8, 2026

(54) EQUIPMENT FOR SIMULTANEOUSLY TESTING REFERENCE AND TEST NASAL SPRAY PRODUCTS

(71) Applicant: Brio Pharmaceuticals Inc., Houston, TX (US)

(72) Inventors: Julia Ta, Houston, TX (US); Jose Mejia, Katy, TX (US); Mohana Rao Manam, Missouri City, TX (US); Aarush Gelli, Irving, TX (US); Rahul Amarneni, Ashburn, VA (US); Rayha Manam, Round Rock, TX (US); Rajesh Chawla, Richmond, TX (US); Sai Tharuni Kethamreddy, Houston, TX (US)

(21) Appl. No.: 19/638,828

(22) Filed: Apr. 3, 2026

Related U.S. Application Data
(60) Provisional application No. 63/784,804, filed on Apr. 7, 2025.

(51) Int. Cl. A61M 15/00

(52) U.S. Cl. CPC A61M 15/009; A61M 2205/3306; A61M 2205/70; A61M 2209/02

(57) ABSTRACT

The present disclosure relates to equipment for testing reference and test nasal spray products, and specifically relates to equipment for testing reference and test nasal spray products simultaneously.

### CROSS-REFERENCE TO RELATED APPLICATION

This application claims priority to U.S. Provisional Application No. 63/784,804, filed on Apr. 07, 2025 the contents of which are incorporated herein by reference

### TECHNICALFIELD

The present disclosure relates to equipment for testing reference and test nasal spray products.

### BACKGROUND

Nasal spray devices are used to deliver both locally and systemically acting pharmaceutical products, and are readily available in the marketplace in unit dose and metered multi-dose formats.

Several nasal sprays have been introduced as a new dosage form of administration for quicker onset of action and preventing first pass metabolism. The regulatory agencies issued several guidances for establishing equivalency of generic products with those of the branded products.

Two drug products are considered to be bioequivalent if there is no significant difference in the rate or extent at which equal doses of an active ingredient becomes available at its site of action under similar conditions.

The parameters insisted by regulatory agencies for establishing in vitro bioequivalence are single actuation content through container life, droplet size distribution by laser diffraction, drug in small particles/droplets by cascade impactor, spray pattern, plume geometry, priming and repriming, drug particle size distribution by microscopy for suspensions in addition to the Q1 and Q2 equivalency.

### SUMMARY

This disclosure describes example techniques to provide equipment for testing reference and test nasal spray products. This disclosure provides for equipment for simultaneously testing reference and test nasal spray products. This disclosure also provides for equipment for simultaneously testing reference and test nasal spray products using thin-layer chromatography (TLC) technique.

This disclosure describes example techniques to provide equipment with synchronized actuation mechanism with single force-distribution plate, TLC integration enabling simultaneous deposition + imaging correlation, unique dual-zone chamber design preventing cross-interference.

The present disclosure specifically relates to an equipment for simultaneously testing reference and test nasal spray products. It is considered useful to design an equipment for testing both the reference and test products simultaneously in one equipment to avoid inconsistencies in results. Simultaneous testing of nasal spray products reference and test formulations requires equipment and systems designed to reduce variances in testing due to human error, equipment calibration, or environmental conditions.

These systems typically include two nozzles that can spray both the reference and test formulations at the same time, ensuring consistent spray characteristics. This system ensures that both products are tested under the same conditions with high repeatability. These systems measure key spray characteristics such as: spray pattern (e.g., angle, droplet size distribution, and plume geometry), spray volume (dose delivered per actuation), spray velocity and delivery consistency (time between sprays, force applied, etc.).

This disclosure describes examples of equipment for testing reference and test nasal spray products simultaneously. The equipment having (e.g., essentially having) different parts to operate the both products at same time.

In some examples, the disclosure relates to an equipment for simultaneously testing reference and test nasal spray products using TLC technique.

Accordingly, the disclosure provides equipment for testing reference and test nasal spray products.

In one aspect, equipment for simultaneously testing reference and test nasal spray products using TLC technique is described.

In one aspect, the equipment comprise nasal spray pump holders (1), actuating plate (2), dividing wall (3), holders for TLC plates with slots (4), laser that can be adjusted and can be rotated horizontally for spray pattern or vertically for plume geometry (5), cameras for plume geometry (6), cameras for spray pattern (7), doors (8), adjustable base and controller to angle device (9) and trigger for actuation and cameras (10).

In one aspect, an equipment for simultaneously testing a reference nasal spray product and a test nasal spray product, comprising:

a housing defining an enclosed testing chamber;

a first holder configured to retain the reference nasal spray product;

a second holder configured to retain the test nasal spray product, the first and second holders being aligned to direct sprays toward one or more measurement zones;

a synchronized actuation mechanism configured to actuate the reference nasal spray product and the test nasal spray product substantially simultaneously with controlled force;

a deposition measurement system comprising one or more substrate holders positioned at predetermined distances from the sprays;

an optical measurement system comprising at least one laser and at least one camera configured to capture spray characteristics; and

a controller configured to synchronize actuation and data acquisition.

In one aspect, the synchronized actuation mechanism comprises a single actuating plate configured to actuate both spray products simultaneously.

In one aspect, the deposition measurement system comprises holders configured to receive thin-layer chromatography (TLC) plates for determination of single actuation content.

In one aspect the substrate holders are positioned at distances of approximately 3 cm, 5 cm, and 7 cm from the spray nozzles.

In one aspect, the equipment further comprises an adjustable base configured to vary the angle of the spray products.

In one aspect, a method for simultaneously testing a reference nasal spray product and a test nasal spray product, comprising:

positioning the reference and test nasal spray products in aligned holders;

simultaneously actuating both products using a synchronized actuation mechanism;

capturing spray plume geometry and spray pattern using an optical measurement system; and

collecting deposition on one or more substrates for comparative analysis.

In one aspect, the equipment comprises a housing defining an enclosed testing chamber, one or more spray device holders, an automated actuating mechanism, adjustable measurement stations for deposition analysis, optical measurement systems including lasers and cameras and a synchronization controller.

In one embodiment, the equipment enables simultaneous capture of spray plume geometry and spray pattern, adjustable measurement distances (e.g., 3 cm, 5 cm, 7 cm), controlled actuation and synchronized imaging and modular and dual-chamber testing configurations.

### BRIEFDESCRIPTIONOFDRAWINGS

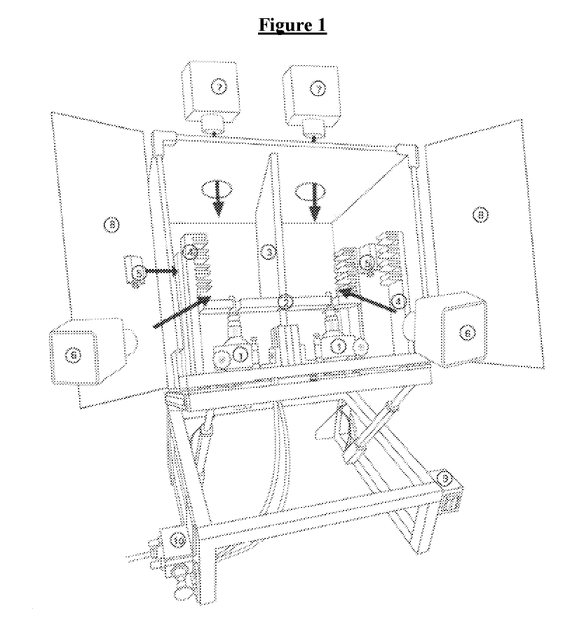

FIG. 1: Front-view of nasal spray testing equipment.

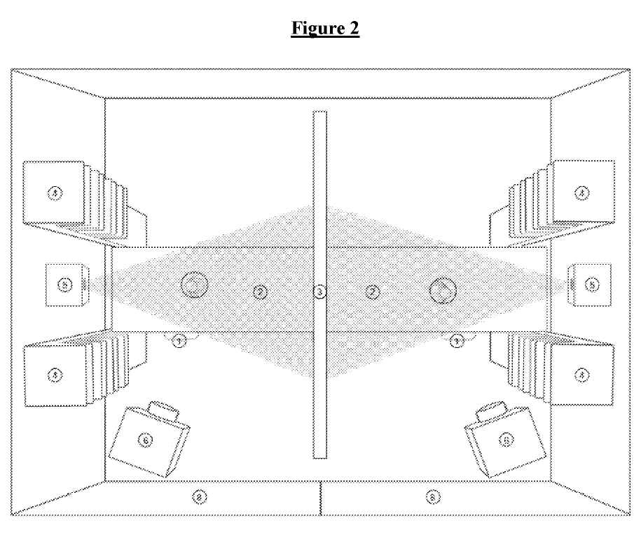

FIG. 2: Top view of nasal spray products testing equipment.

### DETAILEDDESCRIPTION

Accordingly, the disclosure provides equipment for testing reference and test nasal spray products. As described, in one aspect, equipment for testing reference and test nasal spray products using TLC technique.

Several equipment are available for testing the characters of brand product (reference) and its generic version(test) to establish in vitro bioequivalence similarity between the two. Presently available equipment test the reference and test products separately. There is possibility of operator-related or equipment and its handling related issues, which may contribute to inconsistencies in results.

Accurate characterization of spray devices, particularly nasal spray pumps, is used for pharmaceutical development and regulatory compliance. Existing equipment measure plume geometry and spray pattern of test and reference products separately, which may introduce variability.

This disclosure describes examples of an integrated equipment that enables simultaneous, repeatable, and configurable measurement of spray plume geometry and deposition patterns under controlled conditions. For example, the disclosure describes examples of an equipment for testing reference and test nasal spray products simultaneously which has several advantages over the existing methods.

Thin-layer chromatography (TLC) is an analytical technique that can be effectively applied to test nasal spray formulations, particularly when assessing the chemical composition, active ingredient quantification, and bioequivalence between a reference and test nasal spray product. The technique involves separating compounds from the formulation based on their interactions with a stationary phase (TLC plate) and a mobile phase (solvent). TLC can be useful for in vitro analysis, as it can provide qualitative and quantitative data about the active ingredients and any other compounds present in the nasal spray.

There may be various advantages of TLC in nasal spray testing, such as but not limited to or requiring all: simplicity and cost-effectiveness: TLC is an affordable and relatively simple technique compared to other methods like HPLC or mass spectrometry; quick results: TLC results can be obtained relatively quickly, providing faster feedback for comparative analysis; sensitive to active ingredients: TLC can effectively detect even small amounts of active pharmaceutical ingredients, which is essential for evaluating nasal sprays; simultaneous analysis: with proper equipment (e.g., automatic applicators, densitometers), possible to test both the reference and test formulations at the same time, reducing operator error and variability.

According to one aspect, the simultaneous testing may give the following benefits, or more or less than the following benefits: reduction in variability: by testing both products at the same time, variability from operator actions may be reduced or eliminated, equipment setup, and environmental changes; improved reproducibility: simultaneous testing enhances reproducibility by standardizing conditions for both reference and test products; faster testing: it can reduce overall testing time since both products are tested in parallel, which may be beneficial for batch consistency analysis.

By employing these automated systems, it may be possible to reduce operator error and variability and achieve a higher degree of consistency and accuracy in nasal spray product testing.

In one aspect, equipment for testing reference and test nasal spray products comprising nasal spray pump holders (1), actuating plate (2), dividing wall (3), holders for TLC plates with slots (4), laser that can be adjusted and can be rotated horizontally for spray pattern or vertically for plume geometry (5), cameras for plume geometry (6), cameras for spray pattern (7), doors (8), adjustable base and controller to angle device (9) and trigger for actuation and cameras (10).

Nasal spray pump holders (1), to hold the reference and test pumps at same level for simultaneous actuation.

Actuating plate (2), for synchronized actuation of both reference and test pumps.

Dividing wall (3), for preventing cross interference of the laser beam.

Holders for TLC plates with slots (4), for collection of spray for estimation of the single actuation content of the reference and test product simultaneously.

Laser (5) that can be adjusted from 3 to 7 cm and can be rotated horizontally for spray pattern or vertically for plume geometry.

Cameras for plume geometry (6), to capture angle and width of the plume.

Cameras for spray pattern (7), to capture Dₘᵢₙ, Dₘₐₓ, ovality, perimeter, and area of the spray.

Doors (8), to prevent interference from the outside environment and protect users from exposure to the laser beam.

Adjustable base and controller to angle device (9) to measure quantify the spray deliverability relative to the angle of handling.

Trigger for actuation and cameras (10) to ensure synchronized activation.

The equipment includes two nasal spray pump holders (1) configured to securely hold nasal spray devices in a fixed orientation. An actuating plate (2) is positioned to apply a controlled force to actuate the spray pump, ensuring repeatable discharge conditions.

A dividing wall (3) separates different sections of the equipment to minimize interference between plume visualization and deposition measurement zones.

Holders for thin-layer chromatography (TLC) plates (4) are provided with slots to enable accurate placement and alignment of deposition substrates.

A laser unit (5) is configured to be both adjustable and rotatable. In a first configuration, the laser is oriented horizontally to illuminate and analyze the spray pattern. In a second configuration, the laser is oriented vertically to visualize plume geometry.

Two cameras for plume geometry (6) are positioned to capture images of the spray plume as it develops in space. Additionally, two cameras to capture spray pattern (7).

The system may be enclosed by doors (8) to reduce environmental disturbances such as airflow and ambient light variations.

An adjustable base with controller (9) allows the equipment to be positioned at different angles, simulating real-world usage conditions.

A trigger mechanism (10) is configured to simultaneously actuate the spray pump and initiate image capture from the cameras, ensuring synchronization and reproducibility.

According to one aspect, the equipment includes an enclosure defining a controlled chamber. The enclosure may include hinged doors to allow access while maintaining environmental control during operation.

One or more spray pump holders are configured to securely retain spray devices in a fixed orientation. The holders may be adjustable to accommodate different container geometries.

An actuating plate is positioned above or in contact with the spray pumps. The actuating plate is driven by a mechanical or electromechanical system to depress the spray pump in a controlled and repeatable manner.

A dividing wall may be positioned within the enclosure to create multiple testing compartments, allowing simultaneous or independent testing.

A plurality of holders are arranged to receive collection substrates, such as thin-layer chromatography (TLC) plates. These holders are positioned at predetermined distances from the spray nozzle, including but not limited to 3 cm, 5 cm, and 7 cm.

An adjustable laser system is configured to measure spray characteristics. The laser may translate between discrete positions corresponding to measurement distances, rotate horizontally to assess spray pattern, rotate vertically to assess plume geometry.

The equipment includes at least one camera positioned to capture plume geometry and at least one camera positioned to capture spray pattern.

The cameras may be synchronized with the actuation system to capture time-resolved spray behavior.

The equipment may include an adjustable base configured to vary the angle of the spray device relative to gravity, enabling simulation of different usage conditions.

The Trigger system coordinates:
a) Actuation of the spray pump
b) Activation of cameras
c) Operation of the laser system

We claim:

1. An equipment for simultaneously testing a reference nasal spray product and a test nasal spray product, comprising:
a housing defining an enclosed testing chamber;
a first holder configured to retain the reference nasal spray product;
a second holder configured to retain the test nasal spray product, the first and second holders being aligned to direct sprays toward one or more measurement zones;
a synchronized actuation mechanism configured to actuate the reference nasal spray product and the test nasal spray product substantially simultaneously with controlled force;
a deposition measurement system comprising one or more substrate holders positioned at predetermined distances from the reference nasal spray product and the test nasal spray product;
an optical measurement system comprising at least one laser and at least one camera configured to capture spray characteristics; and
a controller configured to synchronize actuation and data acquisition.

2. The equipment of claim 1, wherein the synchronized actuation mechanism comprises a single actuating plate configured to actuate both the reference nasal spray product and the test nasal spray product simultaneously.

3. The equipment of claim 1, wherein the deposition measurement system comprises holders configured to receive thin-layer chromatography (TLC) plates for determination of single actuation content.

4. The equipment of claim 1, wherein the one or more substrate holders are positioned at distances of approximately 3 cm, 5 cm, and 7 cm from spray nozzles of the reference nasal spray product and the test nasal spray product.

5. The equipment of claim 1, further comprising an adjustable base configured to vary the angle of the reference nasal spray product and the test nasal spray product .

6. An equipment for simultaneously testing a reference nasal spray product and a test nasal spray product, comprising:
Nasal spray pump holders (1),
Actuating plate (2),
Dividing wall (3),
Holders for thin-layer chromatography (TLC) plates with slots (4),
Laser that can be adjusted and can be rotated horizontally for spray pattern or vertically for plume geometry (5),
Cameras for plume geometry (6),
Cameras for spray pattern (7),
Doors (8),
Adjustable base and controller to angle device (9) and
Trigger for actuation and cameras (10).

7. A method for simultaneously testing a reference nasal spray product and a test nasal spray product, comprising:
positioning the reference and test nasal spray products in aligned holders;
simultaneously actuating both products using a synchronized actuation mechanism;
capturing spray plume geometry and spray pattern using an optical measurement system; and
collecting deposition on one or more substrates for comparative analysis.
